(19) United States
(12) Patent Application Publication
(10) Pub. No.: US 2011/0224460 A1
(43) Pub. Date: Sep. 15, 2011

(54) PROCESS FOR MAKING AMINOALCOHOL COMPOUNDS

(76) Inventor: David W. Moore, Hebron, IL (US)

(21) Appl. No.: 13/044,671

(22) Filed: Mar. 10, 2011

Related U.S. Application Data
(60) Provisional application No. 61/313,845, filed on Mar. 15, 2010.

(51) Int. Cl. C07C 209/34; C07C 209/84

(52) U.S. Cl. 564/494

(57) ABSTRACT

Provided is a process for making an aminoalcohol compound. The process comprises using an excess amount of aliphatic aldehyde in a condensation step between the aldehyde and a nitroalkane, and using an aldehyde scavenger in a reductive hydrogenation step. The process yields aminoalcohol compounds exhibiting reduced color and odor.

### CROSS-REFERENCE TO RELATED APPLICATION(S)

This application claims the benefit of priority from U.S. Provisional Patent Application No. 61/313,845, filed Mar. 15, 2010, which application is incorporated by reference herein in its entirety.

### FIELD OF THE INVENTION

The invention relates to a process for making aminoalcohol compounds. The process provides aminoalcohol compounds exhibiting reduced color and odor.

### BACKGROUND OF THE INVENTION

Aminoalcohol compounds play an important role in a variety of commercial and consumer products. For instance, they may be used as neutralizers in paints and coatings or in personal care products.

Aminoalcohol compounds are generally prepared on a commercial scale by a two-step process. The first step is condensation of a nitroalkane compound with an aliphatic aldehyde, such as formaldehyde, to form a nitroalcohol compound. The second is the reductive hydrogenation of the nitroalcohol to the aminoalcohol compound.

The known commercial processes suffer from a number of disadvantages, the primary of which are the formation of undesired byproducts that influence the color and odor of the desired material. It would be an advance in the art, therefore, if new processes were developed that addressed the disadvantages of the known processes.

### BRIEF SUMMARY OF THE INVENTION

The invention provides a process for making an aminoalcohol compound. The process comprises: condensing a nitroalkane compound with an excess of aliphatic aldehyde in the presence of a basic catalyst to form an intermediate product mixture, the intermediate product mixture comprising free aliphatic aldehyde and a nitroalcohol compound; and hydrogenating the intermediate product mixture in the presence of a hydrogenation catalyst and an aldehyde scavenger to form the aminoalcohol compound.

### DETAILED DESCRIPTION OF THE INVENTION

As noted above, the invention provides a process for making aminoalcohol compounds that exhibit various advantages over materials prepared by the conventional process. The process of the invention comprises condensing a nitroalkane with an excess amount of aliphatic aldehyde to form an intermediate product mixture, followed by reductive hydrogenation of the intermediate product mixture in the presence of an aldehyde scavenger. The combination of using excess aldehyde in the condensation step and an aldehyde scavenger in the reductive hydrogenation step, according to the invention, provides an aminoalcohol product that contains reduced levels of impurities and therefore exhibits lower odor and color than conventional materials.

The nitroalkane of the process may be represented by the following formula IV:

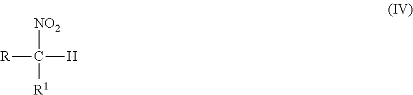

wherein R and R¹ are independently H or C₁-C₆ alkyl (linear or branched).

In some embodiments of the invention, R and R¹ are both H, and the compound is therefore nitromethane.

In some embodiments, R is H and R¹ is C₁-C₆ alkyl, alternatively, C₁-C₅ alkyl, alternatively C₁-C₃ alkyl. In some embodiments, the compound is nitroethane or 1-nitropropane. In a particular embodiment, the compound is 1-nitropropane.

In some embodiments, R and R¹ are independently C₁-C₆ alkyl, alternatively they are C₁-C₃ alkyl, or alternatively they are C₁-C₂ alkyl. In some embodiments, the compound is 2-nitropropane.

The aliphatic aldehyde used in the process may be represented by the formula (III):

R²CHO (III)

wherein R² is H or is C₁-C₆ alkyl (linear or branched). In some embodiments, R² is H. In some embodiments, R² is C₁-C₄ alkyl. In some embodiments, the compound is formaldehyde, alternatively acetaldehyde, alternatively propionaldehyde, or alternatively it is butyraldehyde. In a particular embodiment, the compound is formaldehyde.

In the condensation of a nitroalkane compound with an aliphatic aldehyde, the aldehyde reacts with the hydrogen atoms attached to the nitro-bound carbon atom of the nitroalkane, replacing each such hydrogen with an alkanol substituent. According to the process of the invention, an excess amount of the aliphatic aldehyde is used in the condensation reaction. Use of an excess of the aliphatic aldehyde reduces formation of unwanted side-products, such as lower substituted homologues of the desired product.

By “excess” or “excess amount,” as employed herein in relation to the aliphatic aldehyde, is meant that an amount of the aldehyde is used such that the intermediate product mixture, once the condensation reaction reaches completion, contains free (unreacted) aliphatic aldehyde. Typically, in order to achieve such excess, an amount of the aliphatic aldehyde is used that is greater than required to stoichiometrically react with all of the hydrogen atoms on the nitro-bound carbon of the nitroalkane molecule. For example, if the nitro bound carbon atom contains three hydrogen atoms (i.e., it is nitromethane), greater than three equivalents of the aldehyde are typically used in the process for each equivalent of the nitroalkane. By way of further example, if the nitro bound carbon contains two hydrogen atoms (e.g., 1-nitropropane), greater than two equivalents of aliphatic aldehyde are used. Similarly, if the nitroalkane contains one hydrogen atom at the nitro-bound carbon (e.g., 2-nitropropane), greater than one equivalent of the aliphatic aldehyde is used.

In some embodiments of the process of the invention, the intermediate product mixture, following completion of the condensation reaction, comprises at least about 0.3 weight percent, alternatively at least about 0.4 weight percent, alternatively at least about 0.5 weight percent, alternatively at least about 1 weight percent, alternatively at least about 1.5 weight percent, or alternatively at least about 2 weight percent of free aliphatic aldehyde based on the weight of the nitroalcohol present in the intermediate product mixture. In some embodiments, the amount of free aliphatic aldehyde in the intermediate product mixture is about 6 weight percent or less, alternatively about 4 weight percent or less, or alternatively about 3 weight percent or less, based on the weight of the nitroalcohol. In some embodiments, the amount of free aliphatic aldehyde is between about 1.5 and about 4 weight percent, alternatively between about 2 and about 3 weight percent. In some embodiments, the amount is about 2.5 weight percent.

The condensation reaction is typically conducted in the presence of a basic catalyst. Various basic catalysts may be used including, for example, inorganic bases (e.g., sodium hydroxide, calcium hydroxide) or organic tertiary amines. The tertiary amines are preferred, particularly triethylamine. The concentration of the basic catalyst may be in the range of, for example, 0.2 to 2.0 percent by weight, based on the weight of the nitroalkane.

The condensation reaction may be conducted at elevated temperature, for instance 30 to 80° C., alternatively 40 to 50° C. In a particular embodiment, the temperature is about 50° C. The reaction is continued for sufficient time to permit the desired amount of product to form. Preferably, the reaction is continued until it reaches completion, typically 4 to 8 hours.

The condensation reaction provides, as discussed above, an intermediate product mixture that comprises a nitroalcohol compound and free aliphatic aldehyde. The nitroalcohol compound may be represented by the following formula II:

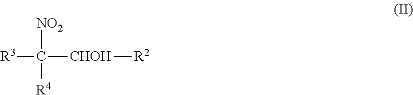

wherein R³ and R⁴ are independently C₁-C₆ alkyl or —CHOH—R², and R² is as defined above.

According to the process of the invention, an aldehyde scavenging agent is combined with the intermediate product mixture and the combination then subjected to a reductive hydrogenation reaction. The hydrogenation reaction converts the nitroalcohol compound to an aminoalcohol compound. The aldehyde scavenger advantageously serves to prevent or mitigate the free aldehyde present in the intermediate product mixture from undergoing undesired side reactions during the hydrogenation step, such as methylation (or alkylation) of the aminoalcohol. As a result, a product that is purer than conventional materials may be achieved.

In some embodiments, the aldehyde scavenger may be an alkylamine compound, such as a C₁-C₆ alkylamine. Examples include ethylamine, propylamine, and butylamine. Preferred is 1-propylamine. In some embodiments, the aldehyde scavenger may be a nitroalkane compound. Typical nitroalkane compounds may be C₁-C₆ nitroalkanes, such as nitroethane, nitropropane, or nitrobutane. The amount of the aldehyde scavenger that is combined with the intermediate product mixture may be at least about 5 mole percent, alternatively at least about 10 mole percent, or alternatively at least 15 mole percent. In some embodiments, the amount is no more than about 40 mole percent, alternatively no more than about 33 mole percent, or alternatively no more than about 28 mole percent, based on the moles of the nitroalcohol compound present in the intermediate product mixture. In some embodiments, the concentration of aldehyde scavenger is between about 15 mole percent and about 28 mole percent. In further embodiments, the concentration is between about 16 and about 20 mole percent. In still further embodiments, the concentration is about 18 mole percent, based on the moles of nitroalcohol in the intermediate product mixture.

The hydrogenation reaction is carried out in the presence of hydrogen gas in combination with a hydrogenation catalyst, for example, Raney nickel or a platinum or palladium based catalyst (Pt or Pd in elemental form or as oxides, with or without supports, e.g., carbon). Preferred is Raney nickel. Conditions for hydrogenation of nitro groups are well known, e.g., a temperature range of about 20-80° C. at a pressure of about 100-1000 psi (690 kPa-6900 kPa) are typical, although these can be readily adjusted by one skilled in the art. The concentration of catalyst may vary, and is typically between about 1 and 25 weight percent, based on the nitroalcohol. A solvent may be used, such as methanol. The hydrogenation reaction is continued until the desired amount of product is formed, preferably to completion, which is typically 1 to 12 hours.

Following reaction, the aminoalcohol product may be filtered to separate it from the catalyst. Additional workup may be carried out, such vacuum removal of excess solvent, and/or distillation of the aminoalcohol.

Optionally, the reaction product of the hydrogenation step may be treated with activated carbon as a further purification step. This optional step may be achieved, for example, by removing low boiling solvents and byproducts from the hydrogenation reaction product, diluting the residue with, for example, water, combining the diluted product with activated carbon, and then stiffing the mixture, e.g., for 1-3 hours. The carbon may then be separated from the product by conventional techniques, such as filtration. Using carbon in this manner serves to further decrease the color and odor of the product aminoalcohol. A preferred material for this optional step is activated carbon having an effective size of 0.6-0.85 mm. Such material may be obtained, for instance, from Siemens, Calgon, or Chemviron.

The aminoalcohol prepared according to the process of the invention may be represented by the following formula I:

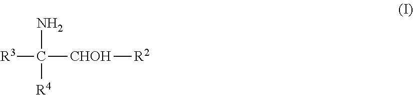

wherein R², R³ and R⁴ are as defined above.

In a particular embodiment of the process of the invention, the starting nitroalkane compound is nitromethane, the aliphatic aldehyde is formaldehyde, and the condensation catalyst is triethylamine. Further, in some embodiments, the amount of free formaldehyde present in the intermediate product mixture is between about 2 and about 3 weight percent, alternatively about 2.5 weight percent, based on the weight of the nitroalcohol. Additionally, in some embodiments, the formaldehyde scavenger of the hydrogenation step is preferably 1-aminopropane at a concentration of between about 15 and about 20, alternatively about 18, mole percent based on the moles of the nitroalcohol compound present in the intermediate product mixture. The nitroalcohol compound resulting from this embodiment is 2-(hydroxymethyl)-2-nitropropane-1,3-diol and the aminoalcohol is 2-amino-2-(hydroxymethyl)propane-1,3-diol.

In a further particular embodiment of the process of the invention, the starting nitroalkane compound is nitroethane, the aliphatic aldehyde is formaldehyde, and the condensation catalyst is triethylamine. Further, in some embodiments, the amount of free formaldehyde in the intermediate product mixture is between about 2 and about 3 weight percent, alternatively about 2.5 weight percent, based on the weight of the nitroalcohol. Additionally, the formaldehyde scavenger of the hydrogenation step is preferably 1-aminopropane at a concentration of between about 15 and about 20, alternatively about 18, mole percent based on the moles of the nitroalcohol compound present in the intermediate product mixture. The nitroalcohol compound resulting from this embodiment is 2-nitro-2-methylpropane-1,3-diol and the aminoalcohol is 2-amino-2-methylpropane-1,3-diol.

In a still further particular embodiment of the process of the invention, the starting nitroalkane compound is 1-nitropropane, the aliphatic aldehyde is formaldehyde, and the condensation catalyst is triethylamine. Further, in some embodiments, the amount of free formaldehyde in the intermediate product mixture is between about 2 and about 3 weight percent, alternatively about 2.5 weight percent, based on the weight of the nitroalcohol. Additionally, the formaldehyde scavenger of the hydrogenation step is preferably 1-aminopropane at a concentration of between about 15 and about 20, alternatively about 18, mole percent based on the moles of the nitroalcohol compound present in the intermediate product mixture. The nitroalcohol compound resulting from this embodiment is 2-nitro-2-ethyl-1,3-propanediol and the aminoalcohol is 2-amino-2-ethyl-1,3-propanediol.

In another particular embodiment of the process of the invention, the starting nitroalkane compound is 2-nitropropane, the aliphatic aldehyde is formaldehyde, and the condensation catalyst is triethylamine. Further, in some embodiments, the amount of free formaldehyde in the intermediate product mixture is between about 0.4 and about 3 weight percent, alternatively about 0.5 weight percent, based on the weight of the nitroalcohol. Additionally, the formaldehyde scavenger of the hydrogenation step is preferably 1-aminopropane at a concentration of between about 5 and about 15, alternatively about 10, mole percent based on the moles of the nitroalcohol compound present in the intermediate product mixture. The nitroalcohol compound resulting from this embodiment is 2-nitro-2-methyl-1-propanol and the aminoalcohol is 2-amino-2-methyl-1-propanol.

Aminoalcohols prepared according to the invention may be used in a variety of applications, such as neutralizers in paints and coatings or in personal care products.

Numeric ranges used in this specification are inclusive of the numbers defining the range. Unless otherwise indicated, ratios, percentages, parts, and the like are by weight.

The following examples are illustrative of the invention but are not intended to limit its scope.

### EXAMPLES

### Examples 1-8

### 2-Amino-2-Ethyl-1,3-Propanediol

Examples 1-8 relate to 2-amino-2-ethyl-1,3-propanediol (AEPD), which may be prepared from 1-nitropropane and formaldehyde).

The analytical (characterization) methods used in the examples are as follows.

GC Analysis. An HP 5890 Series II Gas Chromatograph with a J&W DB-5 column, 30 m*0.25 mm*1.0 μm is used to monitor effects of process changes on GC area %. The FID detector is set at 250° C. and the injector at 180° C. Oven temperature program: 60° C. for 4 minutes, ramp at 30° C./min to 220° C., hold 7 minutes, ramp at 20° C./min to 280° C., and hold 2 minutes. The injection volume was 1 μL with split ratio of 100:1 and helium as carrier gas.

HPLC Analysis. The concentration of 2-nitrobutanol (2-NB), an undesired side product of the condensation reaction, and 2-nitro-2-ethyl-1,3-propanediol (NEPD) are determined by HPLC analysis. Using Waters 2695 Separations Module, HPLC analysis is performed with Alltech OA-1000 size exclusion column. The mobile phase is 0.01 N H₂SO₄ solution. Detection is achieved using Waters 996 Photodiode Array Detector at wave length of 273 nm. Five standard solutions containing both 2-NB and NEPD are prepared for calibration.

Titration Parameters—Percent Free Formaldehyde. The amount of free formaldehyde is determined by reaction and titration. Hydroxylammonium chloride (NH₂OH.HCl) reacts with formaldehyde to form hydrochloric acid. The hydrochloric acid is titrated with sodium hydroxide from which the percent of free formaldehyde is determined. Autotitrator 726 Titroprocessor from Metrohm Ltd. is used for titration. The instrument is calibrated with pH standard solutions at 4, 7, and 10 before titration. 0.1 N NaOH is used as titrant and deionized water as solvent. A blank is analyzed in the same manner as the sample.

Percent Water by Karl Fischer. Water content in the sample is determined by potentiometric detection using volumetric Karl Fischer (KF) titration. Hydranal-Composite 5 is used as titrant and methanol as solvent.

Experimental procedures used in Examples 1-8 are described below.

Nitroalcohol Adjustment (NEPD with varying levels of free formaldehyde). For these studies, commercial NEPD concentrate is analyzed for weight percent (wt/wt) NEPD, 2-NB, and free formaldehyde. In order to understand the effect of excess formaldehyde on the composition of the NEPD product, the following study is conducted. To 10 g samples of NEPD solution is added 0.27, 0.54, 0.81, and 1.08 g of 37% aqueous formaldehyde. The samples are placed in a 40° C. water bath for 2 hours and analyzed. Analytical results are shown in Table 1.

TABLE 1

| | weight percent | | |
|---|---|---|---|
| 37% aqueous | | | free |
| formaldehyde added | NEPD | 2-NB | formaldehyde |
| none | 66.06 | 4.26 | 0.05 |
| 0.27 g | 67.46 | 1.38 | 0.28 |
| 0.54 g | 66.80 | 0.51 | 1.24 |
| 0.81 g | 66.32 | 0.29 | 2.11 |
| 1.08 g | 64.05 | 0.23 | 2.88 |

With this information, the NEPD concentrate is adjusted to a desired formaldehyde level by charging a certain amount of 37% aqueous formaldehyde, mixing, and heating to 40° C. for 2 hours.

Hydrogenation (reduction of NEPD to AEPD). The reactor used for these experiments is a two liter Parr 316 stainless steel autoclave equipped with a Parr model 4842 controller. The system is furnished with internal cooling coils and an external heating mantle for temperature control. The reactor is fitted with a magnetically driven agitator shaft with 3 pitched blade turbine impellers.

The autoclave is charged with 240 g methanol, RANEY® 3111 (a molybdenum promoted RANEY® type nickel catalyst) at 5% loading based on the nitroalcohol feed, and 1-propylamine at 0-28 mole % based on nitroalcohol feed and free formaldehyde level. The autoclave is sealed, pressure purged 3 times with nitrogen (N₂), 3 times with hydrogen (H₂), and then pressured to and regulated at approximately 700 psig H₂. Agitation is begun and set at 600 rpm. Heating is applied until the autoclave temperature reaches 35° C. The cooling water solenoid is set to control the reaction temperature at 55° C.

The nitroalcohol feed is pumped to the reactor using an Eldex Duros model CC-100-S high pressure positive displacement pump. The supply side of the pump is connected to a graduated cylinder and the delivery side is fitted with a relief device. Depending on the amount of free formaldehyde in the NEPD solution (0.05-5.0%), 900-1000 g NEPD solution is pumped to the autoclave. The autoclave contents are sampled for GC analysis at 25, 50, and 75% of the NEPD feed. Following completion of the feed, the contents are held for 10 minutes at constant temperature under hydrogen pressure. The autoclave is then cooled to 25° C., vented, and purged with N₂. The reaction product (final sample) is filtered through a glass microfiber filter to remove catalyst, transferred to glass bottles, and analyzed by GC.

Concentration. A Büchi rotary evaporator, model 011, is used in the laboratory to remove methanol, propylamines, and enough water to produce 2-amino-2-ethyl-1,3-propanediol (AEPD) at 85 concentration. The pressure in the Büchi system is set and controlled at 90 mm Hg using a J-KEM Scientific digital vacuum regulator, model 200. The bottoms temperature (water bath) is slowly increased to 66° C. The system is held at these conditions until the overheads stream nearly stops. Karl Fischer titration shows the product concentrate to contain 13-14% water in each sample processed. At these conditions, the process stops automatically at the target concentration.

The following Examples 1-8 demonstrate the effect of varying the amount of formaldehyde and propylamine (scavenger) on the color and odor of the AEPD product.

### Example 1

900.3 g NEPD solution is used for this example (control). No propylamine is added to the autoclave heel. 1169.3 g autoclave filtrate is recovered.

### Example 2

900.1 g NEPD solution is used for this example. 5 mole % propylamine (14.1 g) is added to the autoclave heel. 1138.3 g autoclave filtrate is recovered.

### Example 3

NEPD adjusted to about 1.25% free formaldehyde with addition of 48.7 g of 37% aqueous formaldehyde to 901.3 g NEPD solution. No propylamine is added to the autoclave heel. 1192.4 g autoclave filtrate is recovered.

### Example 4

NEPD adjusted to about 1.25% free formaldehyde with addition of 48.6 g of 37% aqueous formaldehyde to 900.2 g NEPD solution. 5 mole % propylamine (14.0 g) is added to the autoclave heel. 1201.7 g autoclave filtrate is recovered.

### Example 5

NEPD adjusted to about 1.8% free formaldehyde with addition of 62.5 g of 37% aqueous formaldehyde to 900.0 g NEPD solution. 7 mole % propylamine (21.0 g) is added to the autoclave heel. 1250.2 g autoclave filtrate is recovered.

### Example 6

NEPD adjusted to about 4% free formaldehyde with addition of 72.9 g of 37% aqueous formaldehyde to 900.0 g NEPD solution. 20 mole % propylamine (49.6 g) is added to the autoclave heel. 1270.0 g autoclave filtrate is recovered.

### Example 7

NEPD adjusted to about 5% free formaldehyde with addition of 102.6 g of 37% aqueous formaldehyde to 900.3 g NEPD solution. 28 mole % propylamine (69.8 g) is added to the autoclave heel. 1322.9 g autoclave filtrate is recovered.

### Example 8

NEPD adjusted to about 2% free formaldehyde with addition of 12.6 g of 37% aqueous formaldehyde to 900.0 g NEPD solution. No propylamine is added to the autoclave heel. 1170.0 g autoclave filtrate is recovered.

GC results from examples 1-8 are tabulated in Table 2. In the table, AB refers to 2-aminobutanol, MM means monomethylated derivative, and DM means dimethylated derivative. AB is a lower substituted homologue of the desired material. It is an undesired side product of the condensation reaction that may result from using insufficient amounts of formaldehyde. All MM and DM derivatives are also undesired byproducts that may result when excess formaldehyde is present during reductive hydrogenation of the nitroalcohol.

TABLE 2

| Example | 1 | 2 | 3 | 4 | 5 | 6 | 7 | 8 |
|---|---|---|---|---|---|---|---|---|
| Component | GC area % | | | | | | | |
| AB | 7.14 | 7.15 | 0.93 | 2.50 | 1.60 | 1.52 | 1.89 | 0.30 |
| MMAB | 1.00 | 0.28 | 0.61 | 0.28 | 0.22 | 0.21 | 0.17 | 0.27 |
| DMAB | 0.35 | 0.00 | 1.41 | 0.00 | 0.05 | 0.00 | 0.00 | 1.31 |
| AEPD | 91.20 | 92.28 | 94.33 | 96.78 | 97.30 | 98.00 | 97.34 | 87.99 |
| MMAEPD | 0.16 | 0.00 | 2.46 | 0.16 | 0.42 | 0.11 | 0.19 | 8.20 |
| DMAEPD | 0.00 | 0.00 | 0.00 | 0.00 | 0.00 | 0.00 | 0.00 | 1.17 |
| total | 99.85 | 99.72 | 99.73 | 99.72 | 99.60 | 99.84 | 99.60 | 99.24 |
| area % | 8.48 | 7.43 | 2.94 | 2.78 | 1.87 | 1.73 | 2.06 | 1.88 |
| AB + MMAB + | | | | | | | | |
| DMAB | | | | | | | | |
| area % | 91.36 | 92.28 | 96.78 | 96.94 | 97.73 | 98.11 | 97.53 | 97.36 |
| AEPD + | | | | | | | | |
| MMAEPD | | | | | | | | |
| AEPD′s/AB′s (ratio) | 10.77 | 12.42 | 32.87 | 34.88 | 52.24 | 56.71 | 47.33 | 51.88 |

The results demonstrate that high levels of the undesired AB compound are observed if excess formaldehyde is not used (see examples 1 & 2). With excess formaldehyde but no formaldehyde scavenger in the autoclave, a high amount of methylation takes place, examples 3 & 8. With excess formaldehyde and an appropriate amount of formaldehyde scavenger, dissociation and methylation are significantly decreased, examples 4, 5, 6, & 7.

### Example 9

### Further Purification Using Carbon

This Example illustrates the optional use of carbon for further purification of the aminoalcohol product.

Following removal of methanol and low boiling amines from the autoclave product as described in the examples above, the material is diluted in water and contacted with activated carbon (Weststates brand Aquacarb 1230C from Siemens) for an appropriate time period for removal of color and residual odor. The AEPD product is diluted to 40%, 56.1%, and 86.4% actives in water and 60 g of the diluted AEPD transferred to each of three 125 ml Erlenmeyer flasks equipped with a magnetic stir bar. 10 g of the above activated carbon is added to the corresponding flasks, 1, 2, and 3. The flasks are placed on a magnetic stir plate, mixed for 2 hours at ambient temperature, filtered, and transferred to 4 ounce glass jars. Along with color, the residual odor is removed with carbon treatment and effectiveness is a function of dilution. Color removal results are made be physical observation. Residual odor is believed to be linked to a trace high boiling diamine in the AEPD. Odor removal therefore can be measured by GC with reduction in the amount of the diamine impurity, as shown in Table 3.

TABLE 3

| AEPD Concentration in | Diamine impurity |
|---|---|
| water | GC area percent |
| 40% | not detected |
| 56.1% | 0.037 |
| 86.4% | 0.103 |

### Examples 10-12

### 2-Amino-2-Methyl-1-Propanol

Examples 10-12 relate to 2-amino-2-methyl-1-propanol (AMP), which may be prepared from 2-nitropropane and formaldehyde. In the reaction, one mole of formaldehyde reacts with one mole of the 2-nitropropane to form 2-nitro-2-methyl-1-propanol (NMP), which is reduced to 2-amino-2-methyl-1-propanol (AMP) under conditions as described above.

The NMP of these examples contains 0.57 wt % free formaldehyde. The hydrogenation procedure is similar to that described above except 1-propylamine is used at 0-10 mole %, based on nitroalcohol feed.

### Example 10

950.0 g NMP solution is used for this example (control). No propylamine is added to the autoclave heel. 1131.2 g autoclave filtrate is recovered.

### Example 11

920.0 g NMP solution is used for this example. 5 mole % propylamine (27.5 g) is added to the autoclave heel. 1269.0 g autoclave filtrate is recovered.

### Example 12

920.0 g NMP solution is used for this example. 10 mole % propylamine (55.0 g) is added to the autoclave heel. 1316.10 g autoclave filtrate is recovered.

GC results from examples 10-12 are tabulated in Table 4. Isopropylamine, methylisopropylamine, dimethylisopropylamine, MMAMP, DMAMP, AB, AMPD (2-amino-2-methyl-1,3-propanediol), and AEPD are undesired side products in this example whose minimization is advantageous.

TABLE 4

| | example | | |
|---|---|---|---|
| | 10 | 11 | 12 |
| Component | GC area % | | |
| isopropylamine | 2.51 | 1.01 | 1.07 |
| methylisopropylamine | 1.20 | 0.24 | 0.24 |
| dimethylisopropylamine | 0.71 | 0.00 | 0.00 |
| AMP | 89.28 | 96.56 | 96.77 |
| MMAMP | 3.84 | 0.47 | 0.17 |
| DMAMP | 0.21 | 0.00 | 0.00 |
| AB | 0.04 | 0.04 | 0.05 |
| AMPD | 0.44 | 0.45 | 0.47 |
| AEPD | 0.80 | 0.83 | 0.84 |
| total of major components | 99.04 | 99.60 | 99.61 |
| total isopropylamines | 4.43 | 1.25 | 1.31 |
| AMP + MMAMP + DMAMP | 93.33 | 97.03 | 96.94 |
| degree of methylation* | 4.34% | 0.48% | 0.18% |

*MMAMP + DMAMP/AMP + MMAMP + DMAMP

The results demonstrate that undesired methylation is significantly decreased with presence of a formaldehyde scavenger. Unexpectedly, dissociation, as measured by total isopropylamine, is also reduced, examples 11 & 12.

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A process for making an aminoalcohol compound, the process comprising:
(a) condensing a nitroalkane compound of formula IV: 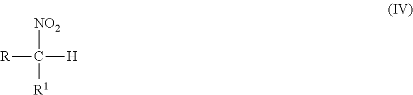
with an excess of aliphatic aldehyde of formula III:
R²CHO (III)
in the presence of a basic catalyst to form an intermediate product mixture, the intermediate product mixture comprising free aliphatic aldehyde of formula III and a nitroalcohol compound of formula (II): 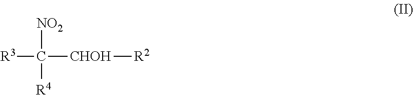
wherein R, R¹, and R² are independently H or C₁-C₆ alkyl, and R³ and R⁴ are independently C₁-C₆ alkyl or —CHOH—R²; and
(b) hydrogenating the intermediate product mixture in the presence of a hydrogenation catalyst and an aldehyde scavenger to form an aminoalcohol compound.

2. The process of claim 1 wherein R and R¹ are both H.

3. The process of claim 1 wherein R is H and R¹ is C₁-C₆ alkyl.

4. The process of claim 1 wherein R and R¹ are independently C₁-C₆ alkyl.

5. The process of claim 1 wherein the nitroalkane compound of formula IV is nitromethane, nitroethane, 1-nitropropane, or 2-nitropropane.

6. The process of claim 1 wherein the intermediate product mixture comprises at least about 0.3 weight percent and no more than about 6 weight percent or less of free aliphatic aldehyde based on the weight of the nitroalcohol present in the intermediate product mixture.

7. The process of claim 1 wherein the aldehyde scavenger is an alkylamine compound or a nitroalkane compound.

8. The process of claim 1 wherein the aldehyde scavenger comprises at least about 5 mole percent and no more than about 40 mole percent based on the moles of the nitroalcohol compound present in the intermediate product mixture.

9. The process of claim 1 further comprising treating the aminoalcohol with activated carbon.

10. The process of claim 1 wherein the aminoalcohol compound is 2-amino-2-(hydroxymethyl)propane-1,3-diol, 2-amino-2-methylpropane-1,3-diol, 2-amino-2-ethyl-1,3-propanediol, or 2-amino-2-methyl-1-propanol.
